(19) United States
(12) Patent Application Publication
(10) Pub. No.: US 2023/0143605 A1
(43) Pub. Date: May 11, 2023

(54) KIT AND SYSTEM FOR EVALUATING GLIOMA AND/OR GASTRIC ADENOCARCINOMA PROGNOSIS

(71) Applicant: Beijing Neurosurgical Institute, Beijing (CN)

(72) Inventors: Tao JIANG, Beijing (CN); Jing CHEN, Beijing (CN); Ruichao CHAI, Beijing (CN); Zheng ZHAO, Beijing (CN)

(73) Assignee: Beijing Neurosurgical Institute, Beijing (CN)

(21) Appl. No.: 17/919,711

(22) PCT Filed: Apr. 20, 2021
(86) PCT No.: PCT/CN21/88478

§ 371 Date: Oct. 18, 2022

(30) Foreign Application Priority Data
Apr. 21, 2020 (CN) 202010318352.7

(51) Int. Cl. C12Q 1/6886

(52) U.S. Cl. CPC C12Q 1/6886; C12Q 1/6844

(57) ABSTRACT

Provided are a kit and system for evaluating glioma and/or gastric adenocarcinoma prognosis. The kit includes a first primer pair capable of specifically amplifying a cDNA fragment of SEQ ID NO: 1, and/or a first probe capable of specifically hybridizing to the cDNA fragment of SEQ ID NO: 1, wherein the cDNA fragment of SEQ ID NO: 1 at least contains a cDNA sequence at positions 4057-4074, positions 4067-4084 or positions 4064-4079 of SEQ ID NO: 1.

### TECHNICAL FIELD

The disclosure relates to the field of biotechnology, in particular to a kit for evaluating glioma and/or gastric adenocarcinoma prognosis and a system for evaluating glioma and/or gastric adenocarcinoma prognosis.

### BACKGROUND

Glioma is a general name of tumors derived from neuroepithelia, accounting for 40%-50% of craniocerebral tumors, and is the most common primary intracranial tumor. Gliomas are difficult to radically treat, the gliomas often relapse after the operation, and have a poor prognosis. The World Health Organization grading system divides the gliomas into grades I-IV according to the histological characteristics of the glioma, and the prognosis of different grades of gliomas varies.

Accurately evaluating the prognosis of glioma patients has important clinical, scientific and social values. In clinical work, accurate prognosis evaluation can guide doctors to formulate personalized examination and treatment schedules for high-risk patients and help the doctors to formulate reasonable reexamination and follow-up visit plans, thus improving the quality of medical services. In scientific research, accurately assessing the risk level of patients' prognosis can provide an important basis for developing effective treatment schedules for high-risk patients, and can become an important reference for testing the effect of new treatments. From the perspective of society, accurate evaluation of the prognosis of patients can provide scientific survival expectations for patients and their families, guide the patients to comply with treatment plans, avoid excessive medical treatment, reduce family financial pressure, and help to improve the doctor-patient relationship.

At present, the evaluation of glioma prognosis is carried out mainly according to the pathological grade of glioma, but the prognosis of different patients with glioma of a same pathological grade may also vary greatly, and the evaluation of glioma prognosis is still not accurate enough only according to the pathological grade of glioma.

### SUMMARY

The disclosure aims to provide a kit for evaluating glioma and/or gastric adenocarcinoma prognosis and a system for evaluating glioma and/or gastric adenocarcinoma prognosis, capable of further improveing the accuracy of evaluation of glioma and/or gastric adenocarcinoma prognosis.

In a first aspect, the disclosure provides a kit for evaluating glioma and/or gastric adenocarcinoma prognosis, where the kit includes a first primer pair capable of specifically amplifying a cDNA fragment of SEQ ID NO. 1, and/or a first probe capable of specifically hybridizing to the cDNA fragment of SEQ ID NO. 1, where the cDNA fragment of SEQ ID NO: 1 at least contains a cDNA sequence at positions 4057-4074, 4067-4084 or 4064-4079 of SEQ ID NO: 1.

Optionally, the cDNA fragment of SEQ ID NO: 1 at least contains a cDNA sequence at positions 4023-4122 of SEQ ID NO: 1; preferably, the cDNA fragment of SEQ ID NO: 1 refers to a cDNA sequence as shown in SEQ ID NO: 1.

Optionally, the first primer pair contains a first primer as shown in SEQ ID NO: 4 and a second primer as shown in SEQ ID NO: 5, and the sequence of the first probe contains a sequence as shown in SEQ ID NO: 6.

In a second aspect, the disclosure provides a kit for evaluating glioma and/or gastric adenocarcinoma prognosis, where the kit includes a second primer pair capable of specifically amplifying an mRNA fragment of SEQ ID NO. 2, and/or a second probe capable of specifically hybridizing to the mRNA fragment of SEQ ID NO. 2, where the mRNA fragment of SEQ ID NO: 2 at least contains an mRNA sequence at positions 2452-2469, 2464-2481 or 2458-2474 of SEQ ID NO: 2.

Optionally, the mRNA fragment of SEQ ID NO: 2 at least contains an mRNA sequence at positions 2416-2516 of SEQ ID NO: 2; preferably, the mRNA fragment of SEQ ID NO: 2 refers to an mRNA sequence as shown in the SEQ ID NO: 2.

Optionally, the second primer pair contains a third primer as shown in SEQ ID NO: 7 and a fourth primer as shown in SEQ ID NO: 8, and the sequence of the second probe contains a sequence as shown in SEQ ID NO: 9.

In a third aspect, the disclosure provides a kit for evaluating glioma and/or gastric adenocarcinoma prognosis, where the kit includes an antibody for resisting an amino acid fragment of SEQ ID NO. 3, where the amino acid fragment of SEQ ID NO. 3 at least contains an amino acid sequence at positions 750-764 of SEQ ID NO: 3.

Optionally, the amino acid fragment of SEQ ID NO. 3 at least contains an amino acid sequence at positions 722-764 of SEQ ID NO: 3; preferably, the amino acid fragment of SEQ ID NO. 3 refers to an amino acid sequence as shown in SEQ ID NO: 3.

In a fourth aspect, the disclosure provides application of a molecular reagent in preparation of a kit for evaluating glioma and/or gastric adenocarcinoma prognosis, where the molecular reagent includes at least one of (1)-(8) as follows:

(1) a cDNA as shown in SEQ ID NO. 1;

(2) a first primer pair capable of specifically amplifying the cDNA as shown in SEQ ID NO. 1;

(3) a first probe capable of specifically hybridizing to the cDNA as shown in SEQ ID NO. 1;

(4) an mRNA as shown in SEQ ID NO. 2;

(5) a second primer pair capable of specifically amplifying the mRNA as shown in SEQ ID NO. 2;

(6) a second probe capable of specifically hybridizing to the mRNA as shown in SEQ ID NO. 2;

(7) a protein as shown in SEQ ID NO. 3; and

(8) an antibody for resisting the protein as shown in SEQ ID NO. 3.

In a fifth aspect, the disclosure provides a system for evaluating glioma and/or gastric adenocarcinoma prognosis, where the system includes an amplification device, a sequencing device, a computing device and an output device.

The amplification device includes a collection unit and an amplification unit, where the collection unit is used for collecting a template nucleic acid fragment and an amplification primer, and the amplification unit is used for amplifying the template nucleic acid fragment by using the amplification primer to obtain an amplification product.

The sequencing device is used for sequencing the nucleic acid sequence of the amplification product to obtain a sequence of the amplification product.

The computing device includes a memory and a processor, where a computer program is stored in the memory, and the processor is configured to execute the computer program stored in the memory so as to achieve the following determination:

if the sequence of the amplification product contains the cDNA sequence as shown in SEQ ID NO. 1 and/or the mRNA sequence as shown in SEQ ID NO. 2, it is determined that the prognosis of glioma and/or gastric adenocarcinoma corresponding to the template nucleic acid fragment is poor.

The output device is used for outputting a determination result of the computing device.

Through the technical solution, the accuracy of evaluation of glioma and/or gastric adenocarcinoma prognosis is effectively improved in the disclosure.

Other features and advantages of the disclosure will be described in detail in the subsequent specific examples.

### BRIEF DESCRIPTION OF THE DRAWINGS

The drawings are used to provide a further understanding of the disclosure and constitute a part of the description, and are used to explain the disclosure together with the following specific examples, but do not constitute limitations of the disclosure. In the drawings:

FIG. 1 is an agarose gel nucleic acid electrophoretogram of cDNA provided by the examples of the disclosure.

FIG. 2 is a diagram showing Sanger sequencing results of cDNA provided by the examples of the disclosure.

FIG. 3 is a survival curve graph of patients with a full-grade glioma sample provided by the examples of the disclosure.

FIG. 4 is a survival curve graph of patients with an IDH mutated full-grade glioma sample provided by the examples of the disclosure.

FIG. 5 is a survival curve graph of patients with a secondary glioblastoma sample provided by the examples of the disclosure.

FIG. 6 is a survival curve graph of patients with an IDH mutated secondary glioblastoma sample provided by the examples of the disclosure.

FIG. 7 is a survival curve graph of patients with a WHO grade III gastric adenocarcinoma sample provided by the examples of the disclosure.

### DETAILED DESCRIPTION

The specific examples of the disclosure are described in detail below in combination with the drawings. It should be understood that the specific examples described herein are only used to illustrate and interpret the disclosure, but not to limit the disclosure.

In a first aspect, the disclosure provides a kit for evaluating glioma and/or gastric adenocarcinoma prognosis, where the kit includes a first primer pair capable of specifically amplifying a cDNA fragment of SEQ ID NO. 1, and/or a first probe capable of specifically hybridizing to the cDNA fragment of SEQ ID NO. 1, where the cDNA fragment of SEQ ID NO: 1 at least contains a cDNA sequence at positions 4057-4074, 4067-4084 or 4064-4079 of SEQ ID NO: 1.

Preferably, the cDNA fragment of SEQ ID NO: 1 at least contains a cDNA sequence at positions 4023-4122 of SEQ ID NO: 1; preferably, the cDNA fragment of SEQ ID NO: 1 refers to a cDNA sequence as shown in SEQ ID NO: 1.

The cDNA sequence as shown in SEQ ID NO: 1 is cDNA corresponding to a human MET gene with an exon 10 deleted. The inventors of the disclosure have found that when the cDNA can be reversely transcribed from mRNA of glioma and/or gastric adenocarcinoma cells, the malignant degree of glioma and/or gastric adenocarcinoma is higher, and the prognosis of patients is poorer. Where the cDNA fragment of SEQ ID NO: 1 contains a deletion site of the exon 10 of the human MET gene, the cDNA sequence shown in SEQ ID NO: 1 contains all exons, except the exon 10, of the human MET gene, and the sequence shown in SEQ ID NO: 1 is as follows:

tacaagatgttgcatcactttactttaattgcatgatttatcagaacaac
tattaacatacgaagtaccattcagttcagctgcaggtataggcagtgac
aagtatctaattcttagaagaatcacttactcccacaatctgtccagaca
cattaatctaaggacaagtttataaatagcaaacgtgattttcacattgc
agtgttctcaagaatgtatatacaagtgtgtagtcctgttgatgggatgt
ttccccgagttctttctattgatgcgttcatgctcttgaccctggtagag
acagttctttctttccacagagcagattttcttttgtcatccaccattta
caatactctgtgaattagtattacttattacatttatgaaacggcaatat
ttggattgtaatagctcttcagtacaattccttgtgtcttctggtaagtc
tctgcactatcagagagcttttagttataatcattttcctgcaacaacag
ccaaactcaactattgagtttcagtgtgacacaccctctttgtagcttgc
tgggttaacccttggcttcaagtcctgatgatgtaatgagggtggggtgt
attggcaatcagtacatttccttatcgcaatttacagtcattgaaaatca
tgctgtcattaatcccagtctgacataccttttctaaaatgttcacagtg
cagtgtttttgtggcctaacaaaatttttctcatatcattaaaaataaac
atttttataaaaaatataacactttaaatgtttacgtcgacaaaaccagt
tagagtaacctacaccacatgcactatacagtagcaagcacaaaattcca
cagaatgaagcatcacaaagttctgctcagggtggctattccatctaggt
gaaatagctgggattttcaattgcctttttcatttgtttctaaagtatgt
tttgcttaacataaaacacaccctaatgcaaaataaaactccccaaaagt
tttgtttccaattgcttgcgaggtgggaacctgccaccgagacagaggct
aatcttttcaatccatccaccctttctttgctctacctatgagctgtgat
tggaaccaatgaaccttttagtaaaatgtatcctgctttacaaacatgct
gagttatctttaaaaatatttatcaacaaattacttgtcttattttgagt
tttcatttaaaaaaatacacacaaaacatctacatgttcacattcattag
atcagagtagcatcattctcaaacagtgggtttttatatgacaactaaat
atttcatgatgactaaattatttcatacaaattttcttaatgtcatctat
gtacattacatcctgttcatttataaacatgttttgtgtgatgctatttt
gaaactttgactgtattgcctataaatattcttccaaccaaaggaatgtc
tatacaaaaatttataagggctgggcgcagtggctcatgcctgtaatcct
agcactttgggaggctgaggtgggtggatttcttgagtccaggagtttga
aaccagccttggcaacatggcaaaaccccgtctctacaaaaaatacaaaa
attagccggggatggtggtgtgcgcctgtagtcccagctattcgggaggc
ttgagcctgggaggtggaggttgcagtgagctatgatcacaccactgcac
tacagccctggcaacagagtgagatcctgtctcaaaaaacaaacaaacat
ttaaaagggaatgtttacctaatgggtgaatgacaccatcagtaatgaaa
aatgttattgaccatacaacacacaaaagtgttttgaaacactagaattc
taaatgagtggcctgttctggggctgccgctcctgtcctgagcattacta
tctcttctgagttttctgtgatcaagaagccctcaatatttcctgcttca
atttcccatatgaaatcaagtggtacctgatttttaaaaaattcaaccca
gaattcaagttttggactccaatatgacaggagtgttgtcacggctgcag
gccattggtccgtggcctgtgggaccaagcctctggttctgatgctctgt
cagataagaaattccttagaatccagtaatttaaataacaatacaagtcc
tataatagtgcaattttggcaagagcaaagaatatcgatggccttttaaa
ggtcaggcagtgaaaaaaccattggacaaagtgtggactgttgctttgac
atagtactagcactatgatgtctcccagaaggaggctggtcgtgtgtcca
cctcatcatcagcgttatcttctgatgacaacagagaaggatacggagcg
acacattttacgttcacataagtagcgttcacatggacatagtgctcccc
aatgaaagtagagaagatcgctgatatccgggacaccagttcagaaaagg
atgggcgcatttcggctttagggtgccagcattttagcattacttcatat
aaggggtctgggcagtattcgggttgtaggagtcttctcccttgcaacaa
gtaaacagttatatcaaaggtgtttacgtcaggataaggtggggctcctc
ttgtcatcagctcccagaggagcacgccaaaggaccacacatctgacttg
gtggtaaacttttgagtttgcagactttccaaagccatccacttcactgg
cagctttgcacctgttttgttgtgtacactatagtattctttatcataca
tgtctctggcaagaccaaaatcagcaaccttgactgtgaatttttcatcc
agcatacagtttcttgcagccaagtctctgtggacaaactttttgcttgc
aagatatttcatgcctttggctacttgaagaccaaagccaataagatctt
ttacagttggattatgagtctcatttcgaatgaaatttcgaagatctcca
tgtttcatgtatggtaggaccaccagcggagacccttcacttcgcaggca
gattcccaggagcgagaggacattgggatgactaaaatctttcatgatga
ttccctcggtcagaaattgggaaacttctcctatgtcagtgattctgttc
aaggatttcacagcacagtgaattttcttgccatcattgtccaacaaagt
cccatgatatacacaaccaaaatgccctcttcctatgacttcattgaaat
gcacaatcaggctactgggcccaatcactacatgctgcactgcctggacc
agctctggatttagagcactgaggtcaatgtggacagtattttgcagtaa
tggactggatatatcagagtccccactagttaggatgggggacatgtctg
tcagaggatactgcacttgtcggcatgaaccgttctgagatgaattagga
aactgatcttctggaaaagtagctcggtagtctacagattcatttgaaac
catttctgtagttgggcttacacttcgggcacttacaagcctatccaaat
gaggagtgtgtactcttgcatcgtagcgaactaattcactgcccagatct
ttaatttgctttctctttttcagccacaggaaaaacccaagtagtaataa
cagtgctgttgatattgagacaacaccagcaatcaatcctgtgaaattct
gatctggttgaactattacttttccaaggacggttgaagaaattgcttgc
ttccactctatatttagctcgctgttcaatttcagcaggtcattggggac
cgtgcataaaacggcttcagaatgtaagtgtatattctcacagctcttat
ttccaacttttaacacttcacctttaactgcttcagggtcaatatcattt
cccttaatttccagtacattttcattgcccattgagatcatcactggctt
ttcaaaaggcttaaacacaggattatgtacataaatgagatcaaagtatt
tggaaaggatcccatctaacatgaaaaaggctttggttttcagggggagt
tgcagattcagctgttgcagggaaggagtggtacaacagattatctctga
attagagcgatgttgacatgcctaataaaagatttggttggatgaatttc
atagacaatgggatcttcacggtaactgaagatgcttgtctctcggttgg
ctaagtcaattttcaatttaacagcaaactcagttgaaatggtttgggct
ggggtataacattcaagaatactgtttgacacactttttaaagtacatgt
ttttccaccaattgaaatgtgtctagaattcccactgtttaggtaatttc
cagttaaagtaagtaaagtgccaccagccataggaccgtatttcggcgaa
atacttgttattacaggatccacataggagaatgtactgtattgtgttgt
cccgtggccatttgaaataattatggacatattgaaatgcttattcatgg
caggaccaactgtgcatttcaatgtattcatcgtgctctcacttaaagtc
aaggtgcagctctcatttccaaggagaactctagttttctttaaatcaaa
tttattattcctccgaaatccaaagtcccagccacatatggtcagccttg
tccctccttcaaggggtgcactatttgggaaaaccttgtagattgcaggc
agacagatctgttgagtccatgtcccgctcaggcattcctccgatcgcac
acatttgtcgtggcaccagccacactgaacaaagggtggggcagagaggc
attgactgcaggactggaaatgtctgcagcccaagccattcaatgggatc
ttcgtgatcttcttcccagtgataaccagtgtgtagccattttggtttaa
tgtatgctccacaatcacttctggagacactggatgggagtccaggagaa
aattcacatgaggggttgatggtcctgatcgagaaaccacaacctgcatg
aagcgaccctctgatgtcccaagattagctatggtgaggtctcctttaat
gaaggtggatatagatgttaagaggacttcgctgaattgacccatgaata
agtcaacgcgctgcaaagctgtggtaaactctgttcgatattcatcacgg
cgcgcttcacagcctgatgaatttctcagaagtgtcctattaaagcagtg
ctcatgattgggtccgtaaaaatgctggagacatctcacattgtttttgt
tgacgatcttgttgaagaagtcgttgacatatttgatagggaatgcacac
atggcagatcgatccattggttcggcagaatctggcttgctttgtgcgaa
caccccgaaaagaatgtcatcattcaggctggctcctatttgtctagcaa
gctgggccccaggcttgctgacatacgcagcctgaagtatattaaacact
tccttctttgtggatctcttttttctcttttctgtgagaatacactccag
aggcatttccatgtaggaatgcaatccagagtttatggaacagaacctga
ttattcttgtgtgaaaagtctgagcatctagagtttccctttggaccgtc
aagaagtaaataaaattgttgctttcaaaggcatggacatacttaatggg
gtaagaatctctgaactcaggtaaaacatcaatgtaggactggtccgtca
aaaacataaaaccatctttcgtttcctttagccttctcactgatatcgaa
tgcaatggatgatctgggaaataagaagaatttatggtattgcctacaaa
gaagttgatgaaccggtcctttacagatgaaaggactttggctcccaggg
cgctcaccacacagtcaggacactggctgggctcttctatctgtggggag
aatatgcagtgaacctccgactgtatgtcagcagtatgattgtggggaaa
gacatgtcgctggcaggtccctctgttgacgctgccacagctaatgagtt
gatcatcatagtaggtgtcgacaactagagccatgttgatgttatctttc
caaacacctcctgataaattggctttgctgctgcagtcctgacatgggaa
acaatctgggtgttccagcacaggcccagtcttgtactcagcaaccttct
gaaggtcttcctcatttaaaacataaatgtagttagtggcaccaaggaaa
atgtgatgctcatgtagaatgacattctggatgggtgtttccgcggtgaa
gttgggaagctgatacttcatattcacattcatctcggactttgctagtg
cctctttacactccccattgctcctctgcaccaaggtaaacaggagcacg
aggatgccaggtgcaagcacagcgggggccttcattatgagaggtttatc
tttcggtgcccaggaaccagtggagaagtcagcggcgcaaggaccacacg
cgcgctccgcgcctccccgcctcctctcagcaagtcagctgtcgccccgc
atctggctcgcgccctccactcggctccgcatctgctcacaaagcgctcg
gggcgccgcgggcggcgagggcctccgggtcacctgc.

Where the first primer pair only needs to able to specifically amplify the cDNA fragment of SEQ ID NO. 1, and preferably, the first primer pair can contain a first primer as shown in SEQ ID NO: 4 and a second primer as shown in SEQ ID NO: 5. The first probe only needs to be able to specifically hybridize to the cDNA fragment of SEQ ID NO. 1, and preferably, the sequence of the first probe can contain a sequence as shown in SEQ ID NO: 6.

Preferably, the sequence as shown in SEQ ID NO: 4 can be:

atccaaccaaatcttttattaggcatg.

The sequence as shown in SEQ ID NO: 5 can be:

cttctggaaaagtagctcggtagtct.

The sequence as shown in SEQ ID NO: 6 can be:

caaatcttttattaggcatgtcaacatc.

In a second aspect, the disclosure provides a kit for evaluating glioma and/or gastric adenocarcinoma prognosis, where the kit includes a second primer pair capable of specifically amplifying an mRNA fragment of SEQ ID NO. 2, and/or a second probe capable of specifically hybridizing to the mRNA fragment of SEQ ID NO. 2, where the mRNA fragment of SEQ ID NO: 2 at least contains an mRNA sequence at positions 2452-2469, 2464-2481 or 2458-2474 of SEQ ID NO: 2.

Preferably, the mRNA fragment of SEQ ID NO: 2 at least contains an mRNA sequence at positions 2416-2516 of SEQ ID NO: 2; preferably, the mRNA fragment of SEQ ID NO: 2 refers to an mRNA sequence as shown in the SEQ ID NO: 2.

Where the mRNA sequence as shown in SEQ ID NO: 2 is an RNA sequence complementary to the cDNA sequence as shown in SEQ ID NO: 1, and the sequence as shown in SEQ ID NO: 2 is as follows:

gcaggugacccggaggcccucgccgcccgcggcgccccgagcgcuuugug
agcagaugcggagccgaguggagggcgcgagccagaugcggggcgacagc
ugacuugcugagaggaggcggggaggcgcggagcgcgcgugugguccuug
cgccgcugacuucuccacugguuccugggcaccgaaagauaaaccucuca
uaaugaaggcccccgcugugcuugcaccuggcauccucgugcuccuguuu
accuuggugcagaggagcaauggggaguguaaagaggcacuagcaaaguc
cgagaugaaugugaauaugaaguaucagcuucccaacuucaccgcggaaa
cacccauccagaaugucauucuacaugagcaucacauuuuccuuggugcc
acuaacuacauuuauguuuuaaaugaggaagaccuucagaagguugcuga
guacaagacugggccugugcuggaacacccagauuguuucccaugucagg
acugcagcagcaaagccaauuuaucaggagguguuuggaaagauaacauc
aacauggcucuaguugucgacaccuacuaugaugaucaacucauuagcug
uggcagcgucaacagagggaccugccagcgacaugucuuuccccacaauc
auacugcugacauacagucggagguucacugcauauucuccccacagaua
gaagagcccagccaguguccugacuguguggugagcgcccugggagccaa
aguccuuucaucuguaaaggaccgguucaucaacuucuuuguaggcaaua
ccauaaauucuucuuauuucccagaucauccauugcauucgauaucagug
agaaggcuaaaggaaacgaaagaugguuuuauguuuuugacggaccaguc
cuacauugauguuuuaccugaguucagagauucuuaccccauuaaguaug
uccaugccuuugaaagcaacaauuuuauuuacuucuugacgguccaaagg
gaaacucuagaugcucagacuuuucacacaagaauaaucagguucuguuc
cauaaacucuggauugcauuccuacauggaaaugccucuggaguguauuc
ucacagaaaagagaaaaaagagauccacaaagaaggaaguguuuaauaua
cuucaggcugcguaugucagcaagccuggggcccagcuugcuagacaaau
aggagccagccugaaugaugacauucuuuucgggguguucgcacaaagca
agccagauucugccgaaccaauggaucgaucugccaugugugcauucccu
aucaaauaugucaacgacuucuucaacaagaucgucaacaaaaacaaugu
gagaugucuccagcauuuuuacggacccaaucaugagcacugcuuuaaua
ggacacuucugagaaauucaucaggcugugaagcgcgccgugaugaauau
cgaacagaguuuaccacagcuuugcagcgcguugacuuauucauggguca
auucagcgaaguccucuuaacaucuauauccaccuucauuaaaggagacc
ucaccauagcuaaucuugggacaucagagggucgcuucaugcagguugug
guuucucgaucaggaccaucaaccccucaugugaauuuucuccuggacuc
ccauccagugucuccagaagugauuguggagcauacauuaaaccaaaaug
gcuacacacugguuaucacugggaagaagaucacgaagaucccauugaau
ggcuugggcugcagacauuuccaguccugcagucaaugccucucugcccc
acccuuuguucaguguggcuggugccacgacaaaugugugcgaucggagg
aaugccugagcgggacauggacucaacagaucugucugccugcaaucuac
aagguuuucccaaauagugcaccccuugaaggagggacaaggcugaccau
auguggcugggacuuuggauuucggaggaauaauaaauuugauuuaaaga
aaacuagaguucuccuuggaaaugagagcugcaccuugacuuuaagugag
agcacgaugaauacauugaaaugcacaguugguccugccaugaauaagca
uuucaauauguccauaauuauuucaaauggccacgggacaacacaauaca
guacauucuccuauguggauccuguaauaacaaguauuucgccgaaauac
gguccuauggcugguggcacuuuacuuacuuuaacuggaaauuaccuaaa
cagugggaauucuagacacauuucaauugguggaaaaacauguacuuuaa
aaagugugucaaacaguauucuugaauguuauaccccagcccaaaccauu
ucaacugaguuugcuguuaaauugaaaauugacuuagccaaccgagagac
aagcaucuucaguuaccgugaagaucccauugucuaugaaauucauccaa
ccaaaucuuuuauuaggcaugucaacaucgcucuaauucagagauaaucu
guuguaccacuccuucccugcaacagcugaaucugcaacucccccugaaa
accaaagccuuuuucauguuagaugggauccuuuccaaauacuuugaucu
cauuuauguacauaauccuguguuuaagccuuuugaaaagccagugauga
ucucaaugggcaaugaaaauguacuggaaauuaagggaaaugauauugac
ccugaagcaguuaaaggugaaguguuaaaaguuggaaauaagagcuguga
gaauauacacuuacauucugaagccguuuuaugcacgguccccaaugacc
ugcugaaauugaacagcgagcuaaauauagaguggaagcaagcaauuucu
ucaaccguccuuggaaaaguaauaguucaaccagaucagaauuucacagg
auugauugcugguguugucucaauaucaacagcacuguuauuacuacuug
gguuuuuccuguggcugaaaaagagaaagcaaauuaaagaucugggcagu
gaauuaguucgcuacgaugcaagaguacacacuccucauuuggauaggcu
uguaagugcccgaaguguaagcccaacuacagaaaugguuucaaaugaau
cuguagacuaccgagcuacuuuuccagaagaucaguuuccuaauucaucu
cagaacgguucaugccgacaagugcaguauccucugacagacaugucccc
cauccuaacuaguggggacucugauauauccaguccauuacugcaaaaua
cuguccacauugaccucagugcucuaaauccagagcugguccaggcagug
cagcauguagugauugggcccaguagccugauugugcauuucaaugaagu
cauaggaagagggcauuuugguuguguauaucaugggacuuuguuggaca
augauggcaagaaaauucacugugcugugaaauccuugaacagaaucacu
gacauaggagaaguuucccaauuucugaccgagggaaucaucaugaaaga
uuuuagucaucccaauguccucucgcuccugggaaucugccugcgaagug
aagggucuccgcuggugguccuaccauacaugaaacauggagaucuucga
aauuucauucgaaaugagacucauaauccaacuguaaaagaucuuauugg
cuuuggucuucaaguagccaaaggcaugaaauaucuugcaagcaaaaagu
uuguccacagagacuuggcugcaagaaacuguaugcuggaugaaaaauuc
acagucaagguugcugauuuuggucuugccagagacauguaugauaaaga
auacuauaguguacacaacaaaacaggugcaaagcugccagugaagugga
uggcuuuggaaagucugcaaacucaaaaguuuaccaccaagucagaugug
ugguccuuuggcgugcuccucugggagcugaugacaagaggagccccacc
uuauccugacguaaacaccuuugauauaacuguuuacuuguugcaaggga
gaagacuccuacaacccgaauacugcccagaccccuuauaugaaguaaug
cuaaaaugcuggcacccuaaagccgaaaugcgcccauccuuuucugaacu
ggugucccggauaucagcgaucuucucuacuuucauuggggagcacuaug
uccaugugaacgcuacuuaugugaacguaaaaugugucgcuccguauccu
ucucuguugucaucagaagauaacgcugaugaugagguggacacacgacc
agccuccuucugggagacaucauagugcuaguacuaugucaaagcaacag
uccacacuuuguccaaugguuuuuucacugccugaccuuuaaaaggccau
cgauauucuuugcucuugccaaaauugcacuauuauaggacuuguauugu
uauuuaaauuacuggauucuaaggaauuucuuaucugacagagcaucaga
accagaggcuuggucccacaggccacggaccaauggccugcagccgugac
aacacuccugucauauuggaguccaaaacuugaauucuggguugaauuuu
uuaaaaaucagguaccacuugauuucauaugggaaauugaagcaggaaau
auugagggcuucuugaucacagaaaacucagaagagauaguaaugcucag
gacaggagcggcagccccagaacaggccacucauuuagaauucuaguguu
ucaaaacacuuuuguguguuguauggucaauaacauuuuucauuacugau
ggugucauucacccauuagguaaacauucccuuuuaaauguuuguuuguu
uuuugagacaggaucucacucuguugccagggcuguagugcaguggugug
aucauagcucacugcaaccuccaccucccaggcucaagccucccgaauag
cugggacuacaggcgcacaccaccauccccggcuaauuuuuguauuuuuu
guagagacgggguuuugccauguugccaaggcugguuucaaacuccugga
cucaagaaauccacccaccucagccucccaaagugcuaggauuacaggca
ugagccacugcgcccagcccuuauaaauuuuuguauagacauuccuuugg
uuggaagaauauuuauaggcaauacagucaaaguuucaaaauagcaucac
acaaaacauguuuauaaaugaacaggauguaauguacauagaugacauua
agaaaauuuguaugaaauaauuuagucaucaugaaauauuuaguugucau
auaaaaacccacuguuugagaaugaugcuacucugaucuaaugaauguga
acauguagauguuuuguguguauuuuuuuaaaugaaaacucaaaauaaga
caaguaauuuguugauaaauauuuuuaaagauaacucagcauguuuguaa
agcaggauacauuuuacuaaaagguucauugguuccaaucacagcucaua
gguagagcaaagaaaggguggauggauugaaaagauuagccucugucucg
guggcagguucccaccucgcaagcaauuggaaacaaaacuuuuggggagu
uuuauuuugcauuaggguguguuuuauguuaagcaaaacauacuuuagaa
acaaaugaaaaaggcaauugaaaaucccagcuauuucaccuagauggaau
agccacccugagcagaacuuugugaugcuucauucuguggaauuuugugc
uugcuacuguauagugcaugugguguagguuacucuaacugguuuugucg
acguaaacauuuaaaguguuauauuuuuuauaaaaauguuuauuuuuaau
gauaugagaaaaauuuuguuaggccacaaaaacacugcacugugaacauu
uuagaaaagguaugucagacugggauuaaugacagcaugauuuucaauga
cuguaaauugcgauaaggaaauguacugauugccaauacaccccacccuc
auuacaucaucaggacuugaagccaaggguuaacccagcaagcuacaaag
agggugugucacacugaaacucaauaguugaguuuggcuguuguugcagg
aaaaugauuauaacuaaaagcucucugauagugcagagacuuaccagaag
acacaaggaauuguacugaagagcuauuacaauccaaauauugccguuuc
auaaauguaauaaguaauacuaauucacagaguauuguaaaugguggaug
acaaaagaaaaucugcucuguggaaagaaagaacugucucuaccaggguc
aagagcaugaacgcaucaauagaaagaacucggggaaacaucccaucaac
aggacuacacacuuguauauacauucuugagaacacugcaaugugaaaau
cacguuugcuauuuauaaacuuguccuuagauuaaugugucuggacagau
ugugggaguaagugauucuucuaagaauuagauacuugucacugccuaua
ccugcagcugaacugaaugguacuucguauguuaauaguuguucugauaa
aucaugcaauuaaaguaaagugaugcaacaucuugua.

Where the second primer pair only needs to be able to specifically amplify the mRNA fragment of SEQ ID NO. 2, and preferably, the second primer pair can contain a third primer as shown in SEQ ID NO: 7 and a fourth primer as shown in SEQ ID NO: 8. The second probe only needs to be able to specifically hybridize with the mRNA fragment of SEQ ID NO. 2, and preferably, the sequence of the second probe can contain a sequence as shown in SEQ ID NO: 9.

Preferably, the sequence as shown in SEQ ID NO: 7 can be:

taatacgactcactatagggagcgatgttgacatgcctaataaaaga.

The sequence as shown in SEQ ID NO: 8 can be:

gtgtgtcaaacagtattcttg.

The sequence as shown in SEQ ID NO: 9 can be:

ttgacatgcctaataaaagatttggttg.

In a third aspect, the disclosure provides a kit for evaluating glioma and/or gastric adenocarcinoma prognosis, where the kit includes an antibody for resisting an amino acid fragment of SEQ ID NO. 3, where the amino acid fragment of SEQ ID NO. 3 at least contains an amino acid sequence at positions 750-764 of SEQ ID NO: 3.

Preferably, the amino acid fragment of SEQ ID NO. 3 at least contains an amino acid sequence at positions 722-764 of SEQ ID NO: 3; more preferably, the amino acid fragment of SEQ ID NO. 3 refers to an amino acid sequence as shown in SEQ ID NO: 3.

Where the amino acid sequence as shown in SEQ ID NO: 3 is an amino acid sequence encoded by the mRNA sequence as shown in SEQ ID NO: 2, and the amino acid sequence as shown in SEQ ID NO: 3 is as follows:

MKAPAVLAPGILVLLFTLVQRSNGECKEALAKSEMNVNMKYQLPNFTAET
PIQNVILHEHHIFLGATNYIYVLNEEDLQKVAEYKTGPVLEHPDCFPCQD
CSSKANLSGGVWKDNINMALVVDTYYDDQLISCGSVNRGTCQRHVFPHNH
TADIQSEVHCIFSPQIEEPSQCPDCVVSALGAKVLSSVKDRFINFFVGNT
INSSYFPDHPLHSISVRRLKETKDGFMFLTDQSYIDVLPEFRDSYPIKYV
HAFESNNFIYFLTVQRETLDAQTFHTRIIRFCSINSGLHSYMEMPLECIL
TEKRKKRSTKKEVFNILQAAYVSKPGAQLARQIGASLNDDILFGVFAQSK
PDSAEPMDRSAMCAFPIKYVNDFFNKIVNKNNVRCLQHFYGPNHEHCFNR
TLLRNSSGCEARRDEYRTEFTTALQRVDLFMGQFSEVLLTSISTFIKGDL
TIANLGTSEGRFMQVVVSRSGPSTPHVNFLLDSHPVSPEVIVEHTLNQNG
YTLVITGKKITKIPLNGLGCRHFQSCSQCLSAPPFVQCGWCHDKCVRSEE
CLSGTWTQQICLPAIYKVFPNSAPLEGGTRLTICGWDFGFRRNNKFDLKK
TRVLLGNESCTLTLSESTMNTLKCTVGPAMNKHFNMSIIISNGHGTTQYS
TFSYVDPVITSISPKYGPMAGGTLLTLTGNYLNSGNSRHISIGGKTCTLK
SVSNSILECYTPAQTISTEFAVKLKIDLANRETSIFSYREDPIVYEIHPT
KSFIRHVNIALIQR.

Where the antibody for resisting the amino acid fragment of SEQ ID NO. 3 can be a monoclonal antibody and/or a polyclonal antibody.

In a fourth aspect, the disclosure provides application of a molecular reagent in preparation of a kit for evaluating glioma and/or gastric adenocarcinoma prognosis, where the molecular reagent includes at least one of (1)-(8) as follows:

(1) a cDNA as shown in SEQ ID NO. 1;

(2) a first primer pair capable of specifically amplifying the cDNA as shown in SEQ ID NO. 1;

(3) a first probe capable of specifically hybridizing to the cDNA as shown in SEQ ID NO. 1;

(4) an mRNA as shown in SEQ ID NO. 2;

(5) a second primer pair capable of specifically amplifying the mRNA as shown in SEQ ID NO. 2;

(6) a second probe capable of specifically hybridizing to the mRNA as shown in SEQ ID NO. 2;

(7) a protein as shown in SEQ ID NO. 3; and

(8) an antibody for resisting the protein as shown in SEQ ID NO. 3.

In a fifth aspect, the disclosure provides a system for evaluating glioma and/or gastric adenocarcinoma prognosis. The system includes an amplification device, a sequencing device, a computing device and an output device, where the amplification device includes a collection unit and an amplification unit, where the collection unit is used for collecting a template nucleic acid fragment and an amplification primer, and the amplification unit is used for amplifying the template nucleic acid fragment by using the amplification primer to obtain an amplification product; the sequencing device is used for sequencing the nucleic acid sequence of the amplification product to obtain a sequence of the amplification product; the computing device includes a memory and a processor, where a computer program is stored in the memory, and the processor is configured to execute the computer program stored in the memory so as to realize the following determination: if the sequence of the amplification product contains the cDNA sequence as shown in SEQ ID NO. 1 and/or the mRNA sequence as shown in SEQ ID NO. 2, it is determined that the prognosis of glioma and/or gastric adenocarcinoma corresponding to the template nucleic acid fragment is poor; and the output device is used for outputting a determination result of the computing device.

In a sixth aspect, the disclosure provides a method for evaluating glioma and/or gastric adenocarcinoma prognosis, where the method includes a step of detecting whether a to-be-detected glioma and/or gastric adenocarcinoma sample contains the cDNA fragment of SEQ ID NO. 1 and/or the mRNA fragment of SEQ ID NO. 2 and/or the amino acid fragment of SEQ ID NO. 3 as described above. If the to-be-detected glioma and/or gastric adenocarcinoma sample contains the cDNA fragment of SEQ ID NO. 1 and/or the mRNA fragment of SEQ ID NO. 2 and/or the amino acid fragment of SEQ ID NO. 3 as described above, it is indicated that the prognosis of glioma and/or gastric adenocarcinoma patients corresponding to the to-be-detected glioma and/or gastric adenocarcinoma sample is poorer. Where the cDNA and/or the mRNA can be detected by a method of PCR (Polymerase Chain Reaction), RT-RPA (Reverse Transcription-Recombinase Polymerase Amplification), nucleic acid hybridization or high-throughput sequencing, and the protein can be detected by immunoblotting.

The disclosure is further described in detail below by the examples.

### PREPARATION EXAMPLE

This preparation example was used for obtaining glioma samples and obtaining total RNAs and total cDNAs therein.

1211 glioma samples were collected by using operations conforming to the standards of the medical ethical committee, and the pathological characteristics of each glioma sample were determined. Where for each patient from whom a sample was collected, the consent of himself or herself and his or her therapist was obtained before the sample was collected, having a written proof material. Where glioma was diagnosed by utilizing a pathological diagnosis method, the prognosis of the glioma samples was evaluated according to the overall survival of patients corresponding to the glioma samples, and the longer the overall survival of the patients is, the better the prognosis of the glioma is. The characteristics such as the gender, age, pathological grade, tumor category, and overall survival of the patients corresponding to the glioma samples are shown in Table 1.

TABLE 1

| Category | Quantity |
|---|---|
| Total sample number | 1211 |
| World Health Organization pathological grade | |
| Grade II | 328 |
| Grade III | 354 |
| Grade IV | 529 |
| Histopathological classification | |
| Diffuse oligodendroglioma | 110 |
| Diffuse astrocytoma | 193 |
| Diffuse oligoastrocytoma | 25 |
| Anaplastic oligodendroglioma | 99 |
| Anaplastic oligoastrocytoma | 27 |
| Anaplastic astrocytoma | 228 |
| Glioblastoma | 529 |
| Tumor type | |
| Primary | 710 |
| Recurrent | 347 |
| Secondary | 154 |
| Age | |
| Median age | 42 |
| Distribution | 8-79 |
| Gender | |
| Number of males (proportion) | 717 (59.2%) |
| Overall survival | |
| Median survival (95% confidence interval) | 863 (772-1023) |

The total RNAs of the glioma samples were extracted by using a DNA extraction kit (purchased from Qiagen) according to its operation instruction. The total RNAs were detected by an integrity analyzer, and it was confirmed that the RNA integrity number (RIN) of the total RNAs was larger than 7.0. Double-stranded cDNA was synthesized by using a reverse transcription kit (purchased from Invitrogen) according to its operation instruction and by taking the total RNAs as a template.

### EXAMPLE 1

In this example, the cDNAs of the glioma samples synthesized by the preparation example were subjected to PCR verification.

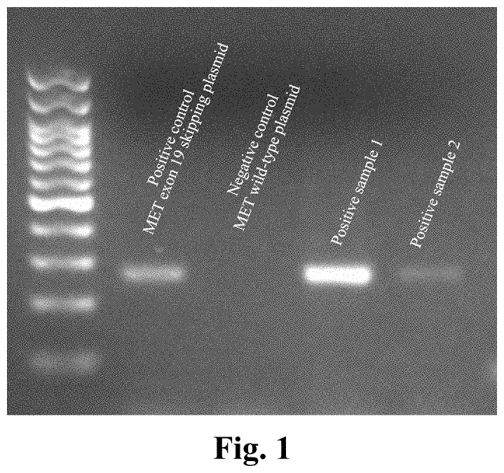

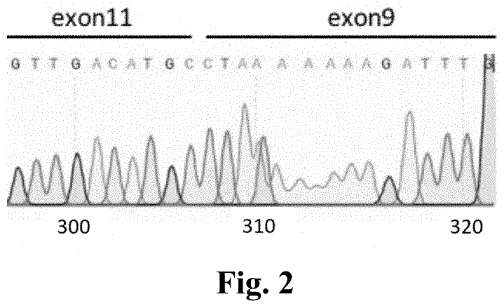

Primers used for the PCR verification were the first primer as shown in SEQ ID NO: 4 and the second primer as shown in SEQ ID NO: 5. The PCR operation was carried out according to the instructions of synthetic primers and the PCR kit. The PCR product was subjected to agarose gel nucleic acid electrophoresis to show whether an amplification band existed or not, as shown in FIG. 1, the appearing amplification bands were recovered by using a DNA gel extraction kit (a QIAquick PCR purification kit, purchased from Qiagen), then Sanger sequencing was performed, and the sequencing result is shown in FIG. 2.

It can be known from FIG. 1 and FIG. 2 that the glioma samples have MET genes with an exon 10 deleted.

### EXAMPLE 2

In this example, RNAs of the glioma samples collected in the preparation example were sequenced.

An RNA library was constructed from RNA of each sample by using a RNA library construction kit (purchased from Illumina), then RNA sequencing was performed on the RNA library by using a sequencing platform (Illumina HiSeq 2000), and glioma samples containing mRNA as shown in SEQ ID NO: 2 were screened from the RNA sequencing results as shown in Table 2.

TABLE 2

| | Glioma samples | Glioma samples |
|---|---|---|
| | with the | with the sequence |
| | sequence of SEQ | of SEQ ID |
| Category | ID NO: 2 detected | NO: 2 undetected |
| Quantity | 14 | 1197 |
| World Health Organization | | |
| pathological grade | | |
| Grade II | 2 | 326 |
| Grade III | 1 | 353 |
| Grade IV | 11 | 518 |
| Histopathological classification | | |
| Diffuse oligodendroglioma | 0 | 110 |
| Diffuse astrocytoma | 2 | 191 |
| Diffuse oligoastrocytoma | 0 | 25 |
| Anaplastic oligodendroglioma | 0 | 99 |
| Anaplastic oligoastrocytoma | 0 | 27 |
| Anaplastic astrocytoma | 1 | 227 |
| Glioblastoma | 11 | 518 |
| Tumor type | | |
| Primary | 6 | 704 |
| Recurrent | 1 | 346 |
| Secondary | 7 | 147 |
| Age | | |
| Median age | 47.5 | 42 |
| Distribution | 29-60 | 8-79 |
| Gender | | |
| Number of males (proportion) | 9 (64.3%) | 708 (59.1%) |
| Overall survival | | |
| Median survival (95% | 200 (96-NA) | 872 (780-1047) |
| confidence interval) | | |

It can be seen from Table 2 that 14 glioma samples containing mRNA shown in SEQ ID NO: 2 were detected with a median survival of 200 days, and 1197 glioma samples not containing the mRNA shown in SEQ ID NO: 2 were detected with a median survival of 872 days, which indicated that the prognosis of glioma containing the mRNA shown in SEQ ID NO: 2 was poor, so that the mRNA can be used for evaluating glioma prognosis.

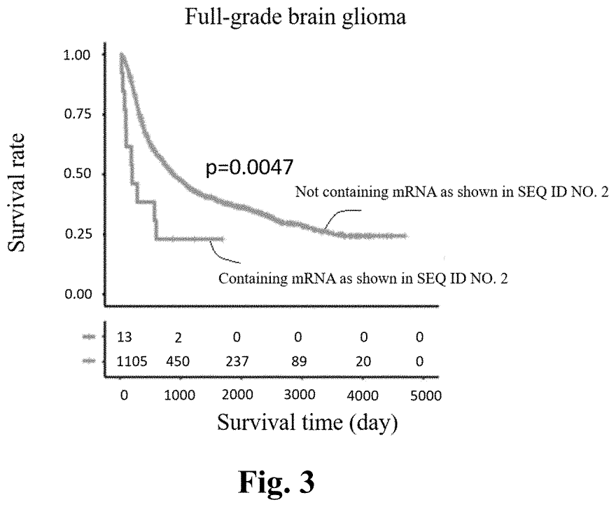

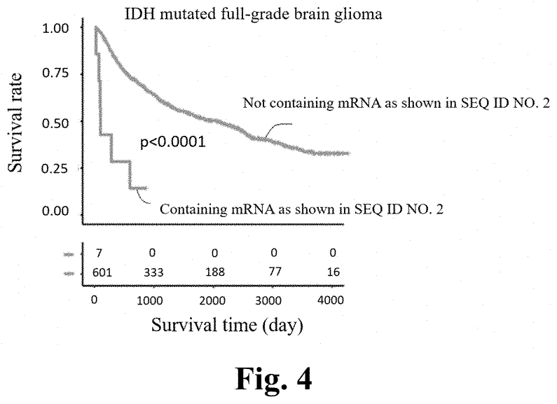

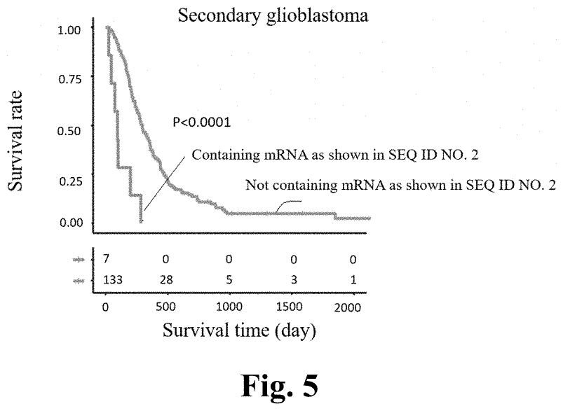

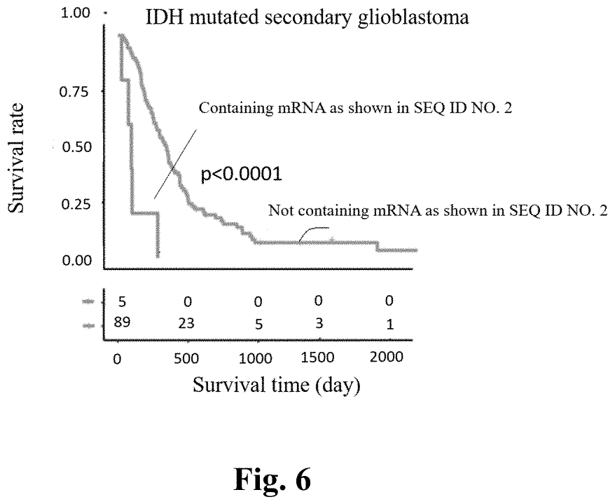

Besides, in the full-grade glioma, the IDH-mutated full-grade glioma, the secondary glioblastoma and the IDH-mutated glioblastoma, the overall survival condition of the glioma samples containing the mRNA shown as the SEQ ID NO: 2 and the overall survival condition of the glioma samples not containing the mRNA shown as the SEQ ID NO: 2 were compared through a survival curve (a Kaplan-Meier curve). Results show that the glioma samples containing the mRNA as shown in SEQ ID NO: 2 were poor in overall survival, as shown in FIGS. 3-6.

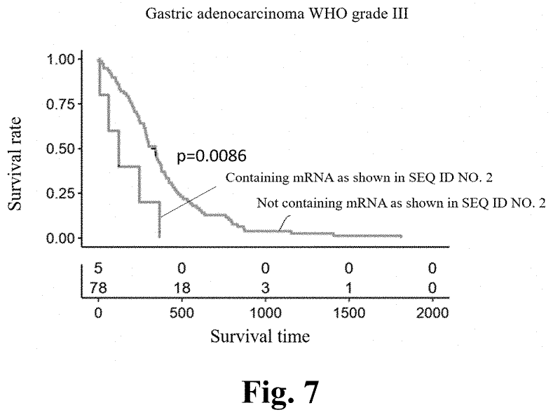

Similarly, it can be proved that the overall survival of patients corresponding to a gastric adenocarcinoma sample containing the mRNA as shown in SEQ ID NO: 2 was poor, and the result is shown in FIG. 7.

The preferred examples of the disclosure are described in detail above in combination with the drawings, however, the disclosure is not limited to the specific details in the above examples, in the technical concept range of the disclosure, the technical solution of the disclosure can be subjected to various simple variations, and these simple variations all belong to the protection range of the disclosure.

In addition, it should be noted that the specific technical features described in the above examples can be combined in any suitable mode without contradiction.

In addition, various different examples of the disclosure can also be combined at will, and as long as the examples do not violate the idea of the disclosure, the examples also should be regarded as the contents disclosed by the disclosure.

1. A kit for evaluating glioma and/or gastric adenocarcinoma prognosis, wherein the kit comprises a first primer pair capable of specifically amplifying a cDNA fragment of SEQ ID NO. 1, and/or a first probe capable of specifically hybridizing to the cDNA fragment of SEQ ID NO. 1, wherein the cDNA fragment of SEQ ID NO: 1 at least contains a cDNA sequence at positions 4057-4074, 4067-4084 or 4064-4079 of SEQ ID NO: 1.

2. The kit according to claim 1, wherein the cDNA fragment of SEQ ID NO: 1 at least contains a cDNA sequence at positions 4023-4122 of SEQ ID NO: 1; preferably, the cDNA fragment of SEQ ID NO: 1 refers to a cDNA sequence as shown in SEQ ID NO: 1.

3. The kit according to claim 2, wherein the first primer pair contains a first primer as shown in SEQ ID NO: 4 and a second primer as shown in SEQ ID NO: 5, and the sequence of the first probe contains a sequence as shown in SEQ ID NO: 6.

4. A kit for evaluating glioma and/or gastric adenocarcinoma prognosis, wherein the kit comprises a second primer pair capable of specifically amplifying an mRNA fragment of SEQ ID NO. 2, and/or a second probe capable of specifically hybridizing to the mRNA fragment of SEQ ID NO. 2, wherein the mRNA fragment of SEQ ID NO: 2 at least contains an mRNA sequence at positions 2452-2469, 2464-2481 or 2458-2474 of SEQ ID NO: 2.

5. The kit according to claim 4, wherein the mRNA fragment of SEQ ID NO: 2 at least contains an mRNA sequence at positions 2416-2516 of SEQ ID NO: 2; preferably, the mRNA fragment of SEQ ID NO: 2 refers to an mRNA sequence as shown in the SEQ ID NO: 2.

6. The kit according to claim 5, wherein the second primer pair contains a third primer as shown in SEQ ID NO: 7 and a fourth primer as shown in SEQ ID NO: 8, and the sequence of the second probe contains a sequence as shown in SEQ ID NO: 9.

7. A kit for evaluating glioma and/or gastric adenocarcinoma prognosis, wherein the kit comprises an antibody for resisting an amino acid fragment of SEQ ID NO. 3, wherein the amino acid fragment of SEQ ID NO. 3 at least contains an amino acid sequence at positions 750-764 of SEQ ID NO: 3.

8. The kit according to claim 7, wherein the amino acid fragment of SEQ ID NO. 3 at least contains an amino acid sequence at positions 722-764 of SEQ ID NO: 3; preferably, the amino acid fragment of SEQ ID NO. 3 refers to an amino acid sequence as shown in SEQ ID NO: 3.

9. (canceled)

10. (canceled)


SEQUENCE LISTING (9 sequences)

SEQ ID NO: 1 - DNA, 6537 bp, Artificial Sequence
  This sequence is synthesized in lab.
tacaagatgttgcatcactttactttaattgcatgatttatcagaacaactattaacatacgaagtaccattcagttcagctgcaggtataggcagtgacaagtatctaattcttagaagaatcacttactcccacaatctgtccagacacattaatctaaggacaagtttataaatagcaaacgtgattttcacattgcagtgttctcaagaatgtatatacaagtgtgtagtcctgttgatgggatgtttccccgagttctttctattgatgcgttcatgctcttgaccctggtagagacagttctttctttccacagagcagattttcttttgtcatccaccatttacaatactctgtgaattagtattacttattacatttatgaaacggcaatatttggattgtaatagctcttcagtacaattccttgtgtcttctggtaagtctctgcactatcagagagcttttagttataatcattttcctgcaacaacagccaaactcaactattgagtttcagtgtgacacaccctctttgtagcttgctgggttaacccttggcttcaagtcctgatgatgtaatgagggtggggtgtattggcaatcagtacatttccttatcgcaatttacagtcattgaaaatcatgctgtcattaatcccagtctgacataccttttctaaaatgttcacagtgcagtgtttttgtggcctaacaaaatttttctcatatcattaaaaataaacatttttataaaaaatataacactttaaatgtttacgtcgacaaaaccagttagagtaacctacaccacatgcactatacagtagcaagcacaaaattccacagaatgaagcatcacaaagttctgctcagggtggctattccatctaggtgaaatagctgggattttcaattgcctttttcatttgtttctaaagtatgttttgcttaacataaaacacaccctaatgcaaaataaaactccccaaaagttttgtttccaattgcttgcgaggtgggaacctgccaccgagacagaggctaatcttttcaatccatccaccctttctttgctctacctatgagctgtgattggaaccaatgaaccttttagtaaaatgtatcctgctttacaaacatgctgagttatctttaaaaatatttatcaacaaattacttgtcttattttgagttttcatttaaaaaaatacacacaaaacatctacatgttcacattcattagatcagagtagcatcattctcaaacagtgggtttttatatgacaactaaatatttcatgatgactaaattatttcatacaaattttcttaatgtcatctatgtacattacatcctgttcatttataaacatgttttgtgtgatgctattttgaaactttgactgtattgcctataaatattcttccaaccaaaggaatgtctatacaaaaatttataagggctgggcgcagtggctcatgcctgtaatcctagcactttgggaggctgaggtgggtggatttcttgagtccaggagtttgaaaccagccttggcaacatggcaaaaccccgtctctacaaaaaatacaaaaattagccggggatggtggtgtgcgcctgtagtcccagctattcgggaggcttgagcctgggaggtggaggttgcagtgagctatgatcacaccactgcactacagccctggcaacagagtgagatcctgtctcaaaaaacaaacaaacatttaaaagggaatgtttacctaatgggtgaatgacaccatcagtaatgaaaaatgttattgaccatacaacacacaaaagtgttttgaaacactagaattctaaatgagtggcctgttctggggctgccgctcctgtcctgagcattactatctcttctgagttttctgtgatcaagaagccctcaatatttcctgcttcaatttcccatatgaaatcaagtggtacctgatttttaaaaaattcaacccagaattcaagttttggactccaatatgacaggagtgttgtcacggctgcaggccattggtccgtggcctgtgggaccaagcctctggttctgatgctctgtcagataagaaattccttagaatccagtaatttaaataacaatacaagtcctataatagtgcaattttggcaagagcaaagaatatcgatggccttttaaaggtcaggcagtgaaaaaaccattggacaaagtgtggactgttgctttgacatagtactagcactatgatgtctcccagaaggaggctggtcgtgtgtccacctcatcatcagcgttatcttctgatgacaacagagaaggatacggagcgacacattttacgttcacataagtagcgttcacatggacatagtgctccccaatgaaagtagagaagatcgctgatatccgggacaccagttcagaaaaggatgggcgcatttcggctttagggtgccagcattttagcattacttcatataaggggtctgggcagtattcgggttgtaggagtcttctcccttgcaacaagtaaacagttatatcaaaggtgtttacgtcaggataaggtggggctcctcttgtcatcagctcccagaggagcacgccaaaggaccacacatctgacttggtggtaaacttttgagtttgcagactttccaaagccatccacttcactggcagctttgcacctgttttgttgtgtacactatagtattctttatcatacatgtctctggcaagaccaaaatcagcaaccttgactgtgaatttttcatccagcatacagtttcttgcagccaagtctctgtggacaaactttttgcttgcaagatatttcatgcctttggctacttgaagaccaaagccaataagatcttttacagttggattatgagtctcatttcgaatgaaatttcgaagatctccatgtttcatgtatggtaggaccaccagcggagacccttcacttcgcaggcagattcccaggagcgagaggacattgggatgactaaaatctttcatgatgattccctcggtcagaaattgggaaacttctcctatgtcagtgattctgttcaaggatttcacagcacagtgaattttcttgccatcattgtccaacaaagtcccatgatatacacaaccaaaatgccctcttcctatgacttcattgaaatgcacaatcaggctactgggcccaatcactacatgctgcactgcctggaccagctctggatttagagcactgaggtcaatgtggacagtattttgcagtaatggactggatatatcagagtccccactagttaggatgggggacatgtctgtcagaggatactgcacttgtcggcatgaaccgttctgagatgaattaggaaactgatcttctggaaaagtagctcggtagtctacagattcatttgaaaccatttctgtagttgggcttacacttcgggcacttacaagcctatccaaatgaggagtgtgtactcttgcatcgtagcgaactaattcactgcccagatctttaatttgctttctctttttcagccacaggaaaaacccaagtagtaataacagtgctgttgatattgagacaacaccagcaatcaatcctgtgaaattctgatctggttgaactattacttttccaaggacggttgaagaaattgcttgcttccactctatatttagctcgctgttcaatttcagcaggtcattggggaccgtgcataaaacggcttcagaatgtaagtgtatattctcacagctcttatttccaacttttaacacttcacctttaactgcttcagggtcaatatcatttcccttaatttccagtacattttcattgcccattgagatcatcactggcttttcaaaaggcttaaacacaggattatgtacataaatgagatcaaagtatttggaaaggatcccatctaacatgaaaaaggctttggttttcagggggagttgcagattcagctgttgcagggaaggagtggtacaacagattatctctgaattagagcgatgttgacatgcctaataaaagatttggttggatgaatttcatagacaatgggatcttcacggtaactgaagatgcttgtctctcggttggctaagtcaattttcaatttaacagcaaactcagttgaaatggtttgggctggggtataacattcaagaatactgtttgacacactttttaaagtacatgtttttccaccaattgaaatgtgtctagaattcccactgtttaggtaatttccagttaaagtaagtaaagtgccaccagccataggaccgtatttcggcgaaatacttgttattacaggatccacataggagaatgtactgtattgtgttgtcccgtggccatttgaaataattatggacatattgaaatgcttattcatggcaggaccaactgtgcatttcaatgtattcatcgtgctctcacttaaagtcaaggtgcagctctcatttccaaggagaactctagttttctttaaatcaaatttattattcctccgaaatccaaagtcccagccacatatggtcagccttgtccctccttcaaggggtgcactatttgggaaaaccttgtagattgcaggcagacagatctgttgagtccatgtcccgctcaggcattcctccgatcgcacacatttgtcgtggcaccagccacactgaacaaagggtggggcagagaggcattgactgcaggactggaaatgtctgcagcccaagccattcaatgggatcttcgtgatcttcttcccagtgataaccagtgtgtagccattttggtttaatgtatgctccacaatcacttctggagacactggatgggagtccaggagaaaattcacatgaggggttgatggtcctgatcgagaaaccacaacctgcatgaagcgaccctctgatgtcccaagattagctatggtgaggtctcctttaatgaaggtggatatagatgttaagaggacttcgctgaattgacccatgaataagtcaacgcgctgcaaagctgtggtaaactctgttcgatattcatcacggcgcgcttcacagcctgatgaatttctcagaagtgtcctattaaagcagtgctcatgattgggtccgtaaaaatgctggagacatctcacattgtttttgttgacgatcttgttgaagaagtcgttgacatatttgatagggaatgcacacatggcagatcgatccattggttcggcagaatctggcttgctttgtgcgaacaccccgaaaagaatgtcatcattcaggctggctcctatttgtctagcaagctgggccccaggcttgctgacatacgcagcctgaagtatattaaacacttccttctttgtggatctcttttttctcttttctgtgagaatacactccagaggcatttccatgtaggaatgcaatccagagtttatggaacagaacctgattattcttgtgtgaaaagtctgagcatctagagtttccctttggaccgtcaagaagtaaataaaattgttgctttcaaaggcatggacatacttaatggggtaagaatctctgaactcaggtaaaacatcaatgtaggactggtccgtcaaaaacataaaaccatctttcgtttcctttagccttctcactgatatcgaatgcaatggatgatctgggaaataagaagaatttatggtattgcctacaaagaagttgatgaaccggtcctttacagatgaaaggactttggctcccagggcgctcaccacacagtcaggacactggctgggctcttctatctgtggggagaatatgcagtgaacctccgactgtatgtcagcagtatgattgtggggaaagacatgtcgctggcaggtccctctgttgacgctgccacagctaatgagttgatcatcatagtaggtgtcgacaactagagccatgttgatgttatctttccaaacacctcctgataaattggctttgctgctgcagtcctgacatgggaaacaatctgggtgttccagcacaggcccagtcttgtactcagcaaccttctgaaggtcttcctcatttaaaacataaatgtagttagtggcaccaaggaaaatgtgatgctcatgtagaatgacattctggatgggtgtttccgcggtgaagttgggaagctgatacttcatattcacattcatctcggactttgctagtgcctctttacactccccattgctcctctgcaccaaggtaaacaggagcacgaggatgccaggtgcaagcacagcgggggccttcattatgagaggtttatctttcggtgcccaggaaccagtggagaagtcagcggcgcaaggaccacacgcgcgctccgcgcctccccgcctcctctcagcaagtcagctgtcgccccgcatctggctcgcgccctccactcggctccgcatctgctcacaaagcgctcggggcgccgcgggcggcgagggcctccgggtcacctgc

SEQ ID NO: 2 - RNA, 6537 nt, Artificial Sequence
  This sequence is synthesized in lab.
gcaggugacccggaggcccucgccgcccgcggcgccccgagcgcuuugugagcagaugcggagccgaguggagggcgcgagccagaugcggggcgacagcugacuugcugagaggaggcggggaggcgcggagcgcgcgugugguccuugcgccgcugacuucuccacugguuccugggcaccgaaagauaaaccucucauaaugaaggcccccgcugugcuugcaccuggcauccucgugcuccuguuuaccuuggugcagaggagcaauggggaguguaaagaggcacuagcaaaguccgagaugaaugugaauaugaaguaucagcuucccaacuucaccgcggaaacacccauccagaaugucauucuacaugagcaucacauuuuccuuggugccacuaacuacauuuauguuuuaaaugaggaagaccuucagaagguugcugaguacaagacugggccugugcuggaacacccagauuguuucccaugucaggacugcagcagcaaagccaauuuaucaggagguguuuggaaagauaacaucaacauggcucuaguugucgacaccuacuaugaugaucaacucauuagcuguggcagcgucaacagagggaccugccagcgacaugucuuuccccacaaucauacugcugacauacagucggagguucacugcauauucuccccacagauagaagagcccagccaguguccugacuguguggugagcgcccugggagccaaaguccuuucaucuguaaaggaccgguucaucaacuucuuuguaggcaauaccauaaauucuucuuauuucccagaucauccauugcauucgauaucagugagaaggcuaaaggaaacgaaagaugguuuuauguuuuugacggaccaguccuacauugauguuuuaccugaguucagagauucuuaccccauuaaguauguccaugccuuugaaagcaacaauuuuauuuacuucuugacgguccaaagggaaacucuagaugcucagacuuuucacacaagaauaaucagguucuguuccauaaacucuggauugcauuccuacauggaaaugccucuggaguguauucucacagaaaagagaaaaaagagauccacaaagaaggaaguguuuaauauacuucaggcugcguaugucagcaagccuggggcccagcuugcuagacaaauaggagccagccugaaugaugacauucuuuucgggguguucgcacaaagcaagccagauucugccgaaccaauggaucgaucugccaugugugcauucccuaucaaauaugucaacgacuucuucaacaagaucgucaacaaaaacaaugugagaugucuccagcauuuuuacggacccaaucaugagcacugcuuuaauaggacacuucugagaaauucaucaggcugugaagcgcgccgugaugaauaucgaacagaguuuaccacagcuuugcagcgcguugacuuauucaugggucaauucagcgaaguccucuuaacaucuauauccaccuucauuaaaggagaccucaccauagcuaaucuugggacaucagagggucgcuucaugcagguugugguuucucgaucaggaccaucaaccccucaugugaauuuucuccuggacucccauccagugucuccagaagugauuguggagcauacauuaaaccaaaauggcuacacacugguuaucacugggaagaagaucacgaagaucccauugaauggcuugggcugcagacauuuccaguccugcagucaaugccucucugccccacccuuuguucaguguggcuggugccacgacaaaugugugcgaucggaggaaugccugagcgggacauggacucaacagaucugucugccugcaaucuacaagguuuucccaaauagugcaccccuugaaggagggacaaggcugaccauauguggcugggacuuuggauuucggaggaauaauaaauuugauuuaaagaaaacuagaguucuccuuggaaaugagagcugcaccuugacuuuaagugagagcacgaugaauacauugaaaugcacaguugguccugccaugaauaagcauuucaauauguccauaauuauuucaaauggccacgggacaacacaauacaguacauucuccuauguggauccuguaauaacaaguauuucgccgaaauacgguccuauggcugguggcacuuuacuuacuuuaacuggaaauuaccuaaacagugggaauucuagacacauuucaauugguggaaaaacauguacuuuaaaaagugugucaaacaguauucuugaauguuauaccccagcccaaaccauuucaacugaguuugcuguuaaauugaaaauugacuuagccaaccgagagacaagcaucuucaguuaccgugaagaucccauugucuaugaaauucauccaaccaaaucuuuuauuaggcaugucaacaucgcucuaauucagagauaaucuguuguaccacuccuucccugcaacagcugaaucugcaacucccccugaaaaccaaagccuuuuucauguuagaugggauccuuuccaaauacuuugaucucauuuauguacauaauccuguguuuaagccuuuugaaaagccagugaugaucucaaugggcaaugaaaauguacuggaaauuaagggaaaugauauugacccugaagcaguuaaaggugaaguguuaaaaguuggaaauaagagcugugagaauauacacuuacauucugaagccguuuuaugcacgguccccaaugaccugcugaaauugaacagcgagcuaaauauagaguggaagcaagcaauuucuucaaccguccuuggaaaaguaauaguucaaccagaucagaauuucacaggauugauugcugguguugucucaauaucaacagcacuguuauuacuacuuggguuuuuccuguggcugaaaaagagaaagcaaauuaaagaucugggcagugaauuaguucgcuacgaugcaagaguacacacuccucauuuggauaggcuuguaagugcccgaaguguaagcccaacuacagaaaugguuucaaaugaaucuguagacuaccgagcuacuuuuccagaagaucaguuuccuaauucaucucagaacgguucaugccgacaagugcaguauccucugacagacaugucccccauccuaacuaguggggacucugauauauccaguccauuacugcaaaauacuguccacauugaccucagugcucuaaauccagagcugguccaggcagugcagcauguagugauugggcccaguagccugauugugcauuucaaugaagucauaggaagagggcauuuugguuguguauaucaugggacuuuguuggacaaugauggcaagaaaauucacugugcugugaaauccuugaacagaaucacugacauaggagaaguuucccaauuucugaccgagggaaucaucaugaaagauuuuagucaucccaauguccucucgcuccugggaaucugccugcgaagugaagggucuccgcuggugguccuaccauacaugaaacauggagaucuucgaaauuucauucgaaaugagacucauaauccaacuguaaaagaucuuauuggcuuuggucuucaaguagccaaaggcaugaaauaucuugcaagcaaaaaguuuguccacagagacuuggcugcaagaaacuguaugcuggaugaaaaauucacagucaagguugcugauuuuggucuugccagagacauguaugauaaagaauacuauaguguacacaacaaaacaggugcaaagcugccagugaaguggauggcuuuggaaagucugcaaacucaaaaguuuaccaccaagucagaugugugguccuuuggcgugcuccucugggagcugaugacaagaggagccccaccuuauccugacguaaacaccuuugauauaacuguuuacuuguugcaagggagaagacuccuacaacccgaauacugcccagaccccuuauaugaaguaaugcuaaaaugcuggcacccuaaagccgaaaugcgcccauccuuuucugaacuggugucccggauaucagcgaucuucucuacuuucauuggggagcacuauguccaugugaacgcuacuuaugugaacguaaaaugugucgcuccguauccuucucuguugucaucagaagauaacgcugaugaugagguggacacacgaccagccuccuucugggagacaucauagugcuaguacuaugucaaagcaacaguccacacuuuguccaaugguuuuuucacugccugaccuuuaaaaggccaucgauauucuuugcucuugccaaaauugcacuauuauaggacuuguauuguuauuuaaauuacuggauucuaaggaauuucuuaucugacagagcaucagaaccagaggcuuggucccacaggccacggaccaauggccugcagccgugacaacacuccugucauauuggaguccaaaacuugaauucuggguugaauuuuuuaaaaaucagguaccacuugauuucauaugggaaauugaagcaggaaauauugagggcuucuugaucacagaaaacucagaagagauaguaaugcucaggacaggagcggcagccccagaacaggccacucauuuagaauucuaguguuucaaaacacuuuuguguguuguauggucaauaacauuuuucauuacugauggugucauucacccauuagguaaacauucccuuuuaaauguuuguuuguuuuuugagacaggaucucacucuguugccagggcuguagugcaguggugugaucauagcucacugcaaccuccaccucccaggcucaagccucccgaauagcugggacuacaggcgcacaccaccauccccggcuaauuuuuguauuuuuuguagagacgggguuuugccauguugccaaggcugguuucaaacuccuggacucaagaaauccacccaccucagccucccaaagugcuaggauuacaggcaugagccacugcgcccagcccuuauaaauuuuuguauagacauuccuuugguuggaagaauauuuauaggcaauacagucaaaguuucaaaauagcaucacacaaaacauguuuauaaaugaacaggauguaauguacauagaugacauuaagaaaauuuguaugaaauaauuuagucaucaugaaauauuuaguugucauauaaaaacccacuguuugagaaugaugcuacucugaucuaaugaaugugaacauguagauguuuuguguguauuuuuuuaaaugaaaacucaaaauaagacaaguaauuuguugauaaauauuuuuaaagauaacucagcauguuuguaaagcaggauacauuuuacuaaaagguucauugguuccaaucacagcucauagguagagcaaagaaaggguggauggauugaaaagauuagccucugucucgguggcagguucccaccucgcaagcaauuggaaacaaaacuuuuggggaguuuuauuuugcauuaggguguguuuuauguuaagcaaaacauacuuuagaaacaaaugaaaaaggcaauugaaaaucccagcuauuucaccuagauggaauagccacccugagcagaacuuugugaugcuucauucuguggaauuuugugcuugcuacuguauagugcaugugguguagguuacucuaacugguuuugucgacguaaacauuuaaaguguuauauuuuuuauaaaaauguuuauuuuuaaugauaugagaaaaauuuuguuaggccacaaaaacacugcacugugaacauuuuagaaaagguaugucagacugggauuaaugacagcaugauuuucaaugacuguaaauugcgauaaggaaauguacugauugccaauacaccccacccucauuacaucaucaggacuugaagccaaggguuaacccagcaagcuacaaagagggugugucacacugaaacucaauaguugaguuuggcuguuguugcaggaaaaugauuauaacuaaaagcucucugauagugcagagacuuaccagaagacacaaggaauuguacugaagagcuauuacaauccaaauauugccguuucauaaauguaauaaguaauacuaauucacagaguauuguaaaugguggaugacaaaagaaaaucugcucuguggaaagaaagaacugucucuaccagggucaagagcaugaacgcaucaauagaaagaacucggggaaacaucccaucaacaggacuacacacuuguauauacauucuugagaacacugcaaugugaaaaucacguuugcuauuuauaaacuuguccuuagauuaaugugucuggacagauugugggaguaagugauucuucuaagaauuagauacuugucacugccuauaccugcagcugaacugaaugguacuucguauguuaauaguuguucugauaaaucaugcaauuaaaguaaagugaugcaacaucuugua

SEQ ID NO: 3 - protein, 764 aa, Artificial Sequence
  This sequence is synthesized in lab.
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFIRHVNIALIQR

SEQ ID NO: 4 - DNA, 27 bp, Artificial Sequence
  This sequence is synthesized in lab.
atccaaccaaatcttttattaggcatg

SEQ ID NO: 5 - DNA, 26 bp, Artificial Sequence
  This sequence is synthesized in lab.
cttctggaaaagtagctcggtagtct

SEQ ID NO: 6 - DNA, 28 bp, Artificial Sequence
  This sequence is synthesized in lab.
caaatcttttattaggcatgtcaacatc

SEQ ID NO: 7 - DNA, 47 bp, Artificial Sequence
  This sequence is synthesized in lab.
taatacgactcactatagggagcgatgttgacatgcctaataaaaga

SEQ ID NO: 8 - DNA, 21 bp, Artificial Sequence
  This sequence is synthesized in lab.
gtgtgtcaaacagtattcttg

SEQ ID NO: 9 - DNA, 28 bp, Artificial Sequence
  This sequence is synthesized in lab.
ttgacatgcctaataaaagatttggttg